(19) United States
(12) Patent Application Publication
(10) Pub. No.: US 2023/0055383 A1
(43) Pub. Date: Feb. 23, 2023

(54) Integrated Data Capture of Sterile and Non-Sterile Surgical Supplies and Implantables

(71) Applicant: Summate Technologies, Inc., Newburyport, MA (US)

(72) Inventors: Philip William Sayles, Newburyport, MA (US); James Tcheng, Durham, NC (US)

(21) Appl. No.: 17/870,488

(22) Filed: Jul. 21, 2022

Related U.S. Application Data
(60) Provisional application No. 63/235,972, filed on Aug. 23, 2021.

(51) Int. Cl. G06K 7/10

(52) U.S. Cl. CPC G06K 7/10544; G06K 7/10821; G06K 7/1404

(57) ABSTRACT

Computer-implemented systems and methods implement a unified workflow to capture data representing the usage of both sterile and non-sterile surgical assets. Such capture may be performed within, or at the edge of, the sterile field. Item usage data about both sterile and non-sterile items may be machine-captured, such as by using one or more of a bar code reader, RFID tag reader, and image capture device. The item usage data about both sterile and non-sterile items may be captured and stored into a single data store by a single software application executing on a single computer, and displayed by that software application in a single user interface. A user may provide input to that software application to certify both the sterile and non-sterile item usage data.

### BACKGROUND

Modern surgical procedures involve the use of a wide variety of items (i.e., supplies and implants), such as screws, plates, Kirschner wires (K-wires), anchors, and drill bits. It is critical to track and create a record of the type and quantity of the supplies and implants used both inside and outside of the surgical field during a particular surgical procedure for a variety of reasons, such as billing, quality assurance, accurate patient records, and determining the type and quantity of items that need to be reordered and restocked. Creating such an accurate record while a surgery is being performed is complicated by a variety of factors, including the wide variety of sterile and non-sterile items that are used, difficulty of tracking what is used inside the sterile field, the lack of space on the tray to print the corresponding part number of each distinct item, the small size of some items, and the difficulty of distinguishing similar items from each other, all in real-time while the surgery is being performed.

An example of a prior art system 400 for tracking use of surgical items is shown in FIG. 4. In such a system 400, traditional manual methods are used for tracking usage of items (i.e., supplies and implants), including both sterile items 402 (which begin outside the sterile field and are brought into the sterile field) and non-sterile items, which are sterilized inside a tray inside the sterile field. Such manual recording methods include, for example, visually identifying sterile items 402 that are used and inputting 406 information about those items 402 into various types of software on a computer outside the sterile field (such as EHR software 408, ERP software 410, and/or OR IS software 412). Such inputting may include for example, scanning the packaging of the items 402 and/or manually inputting information about the items 402 into the software 408, 410, and/or 412. In the case of the non-sterile items 404, such information typically is transcribed 414 manually at the edge of the sterile field and input 416 manually into the software 408, 410, and/or 412.

Manually writing down the type and quantity of such items 402 and 404 on paper or manually keying items into a database, tends to be slow, tedious, and error-prone. Furthermore, optical scanning technology, such as direct part marking using barcodes on the items 402 and 404, has proven to be challenging to implement, due to the nature of the supplies and implants. For example, implants often are made of polished stainless steel, are highly reflective, can be very small, have only irregular surfaces (such as the head of a screw), and are subject to a very harsh reprocessing environment (e.g., heat, steam, and chemicals). All of these factors have made it challenging to directly mark implants. It can also be difficult to read such markings under the bright lights of the surgical field during surgery.

Furthermore, typically there is no computer or other machinery in, or at the edge of, the sterile field for electronically capturing information about item usage at the point of use during surgery. As a result, such capturing typically is performed both manually and outside the sterile field, such as in a corner of the room or even outside of the room containing the sterile field, distant from the actual point of use. The distance, and resulting time delay, between the location of use and the location at which data about the use of items is captured introduces additional opportunities for errors in the item use record.

Errors in the record of the type and quantity of supplies and implants used can have a variety of negative consequences, including failure of the hospital to be reimbursed for all of the items (supplies and implants) that were used, failure to reorder the correct items as a result of inflated inventory levels resulting from failure to accurately record items used in the field, lag time in ordering and restocking items in a timely fashion, and errors in the patient's surgical history which can contribute to suboptimal care for the patient in the future.

What is needed, therefore, are improved techniques for tracking the type and quantity of items used during surgery.

### SUMMARY

Computer-implemented systems and methods implement a unified workflow to capture data representing the usage of both sterile and non-sterile surgical items (i.e., supplies and implants). Such capture may be performed within, or at the edge of, the sterile field. Usage data about both sterile and non-sterile items may be machine-captured, such as by using one or more of a bar code reader, RFID tag reader, and image capture device. The usage data about both sterile and non-sterile items may be captured and stored into a single data store by a single software application executing on a single computer, and displayed by that software application in a single user interface. A user may provide input to that software application to certify both the sterile and non-sterile items usage data.

Other features and advantages of various aspects and embodiments of the present invention will become apparent from the following description and from the claims.

### BRIEF DESCRIPTION OF THE DRAWINGS

FIG. 1 is a diagram of a system for capturing data representing usage of sterile and non-sterile items in a surgical field according to embodiment of the present invention;

FIG. 2 is a flowchart of a method performed by the system of FIG. 1 according to embodiment of the present invention;

FIG. 3 is an illustration of a user interface for displaying captured sterile item and non-sterile item usage data according to embodiment of the present invention; and

FIG. 4 is a diagram of a prior art system for capturing data representing usage of sterile and non-sterile items in a surgical field.

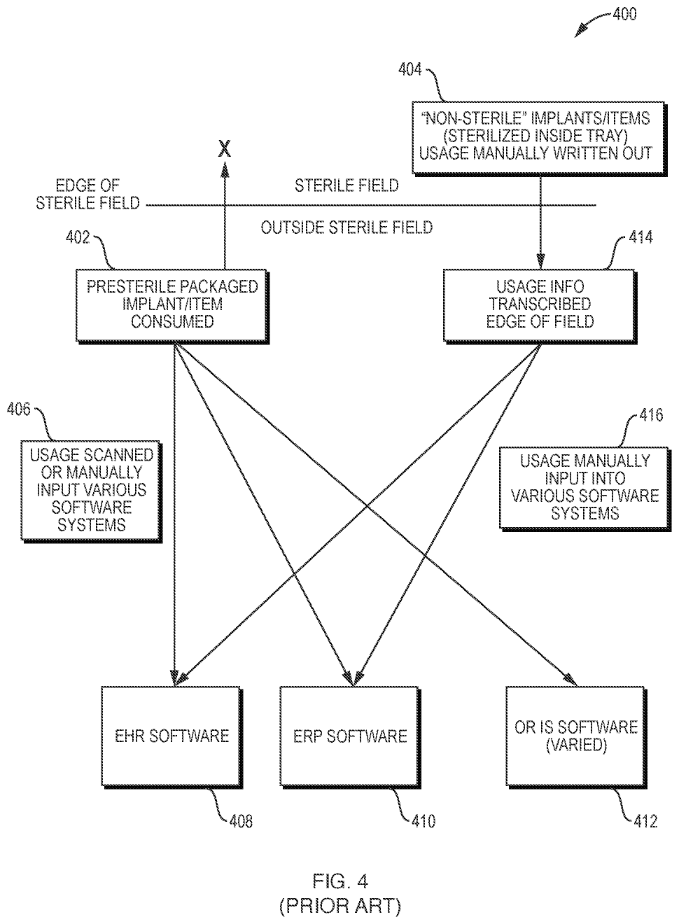

### DETAILED DESCRIPTION

As described above, the state of the art for recording the use of items in the surgical field is to perform such recording manually. (The term “item,” as used herein, includes both supplies and implants, including, but not limited to, any of the specific examples of supplies and implants disclosed herein.) The surgical technician manually reads the micro-printed part number on an item, or on packaging containing the item, or on the tray itself next to the item. In the case of some items, such as tightly-packed screws, there is not enough room to print the part numbers on the tray or the caddy. The part numbers and other characteristics of the items are usually written down on a sterile piece of paper, or on the surgical drape itself. Such manually-written characteristics are then manually matched against a pre-printed inventory sheet outside the sterile field to ascertain the correct part number. This manual process is then followed by a succession of subsequent manual processes, which include manual transcription and data entry steps to complete the recording of each removed asset. The entire process is tedious, time-consuming, and error prone.

Technologies exist for at least partially automating the recording of the use of items in the surgical field. For example, the following patents (referred to herein as “the Summate patents”), which are hereby incorporated by reference herein, describe examples of such technologies:
U.S. Pat. No. 10,470,809, entitled, “Automated Screw Identification System and Method,” issued on Nov. 12, 2019;
U.S. Pat. No. 10,786,331, entitled, “Automated Screw Identification System and Method with Combined Machine-Readable and Human-Readable Markers,” issued on Sep. 29, 2020;
U.S. Pat. No. 10,909,343, entitled, “Automated Screw Identification System and Method with Labeled Pegs,” issued on Feb. 2, 2021.

Embodiments of the present invention provide an improvement to the process of recording item usage. In particular, embodiments of the present invention are directed to systems and methods for recording the usage of both sterile and non-sterile items in a single workflow. Embodiments of the present invention may, for example, store data about the usage of both sterile and non-sterile items in a single data store (e.g., a single software application executing on a single computer).

In general, in the surgical field, there are two categories of surgical items (i.e., supplies and implants): (1) pre-packaged sterile items; and (2) non-sterile items. The packaging which contains the sterile items typically has a machine-readable code thereon (e.g., a bar code). When a sterile item is used (e.g., when the sterile item is removed from its packaging for use in surgery or at some time thereafter), the machine-readable code is read from the packaging of the item. For example, a bar code reader coupled to a first computer outside of the sterile field may be used to read the bar code from the packaging of the sterile item. The code may be used to retrieve additional data about the sterile item. The code and such additional data may be stored in the computer coupled to the bar code reader, such as in application software executing on the first computer.

More specifically, sterile items typically are packaged and gamma-irradiated to sterilize them before they arrive at the hospital. By remaining in their packaging, they remain sterile. Typically, sterile items are placed on a shelf outside the sterile field. When the surgeon requests a particular sterile item, a nurse or other person removes the requested sterile item from the shelf, opens the package outside the sterile field, and passes the sterile item aseptically at the edge of the sterile field to the surgical technician, who takes the sterile item into the sterile field and hands the sterile item to the surgeon. Although sterile items include a variety of artificial objects, sterile items may also include, for example, tissue, such as bone material and skin graft material.

Non-sterile items typically are not pre-packaged in sterile packaging when they arrive at the hospital. They are put into a surgical asset tray, which is put into an autoclave at the hospital, thereby sterilizing the non-sterile items. (Such items are still referred to herein as “non-sterile items” even after they have been sterilized.) The sterilized tray is then put into the sterile field. When the surgeon requests a particular non-sterile item, the surgical technician removes the requested non-sterile item from the tray and hands the requested non-sterile item to the surgeon.

“Consumption” of an item refers to removing the item from its packaging or from the tray that contains it. As the description above implies, sterile items typically are consumed at the edge of the sterile field, while non-sterile items typically are consumed within the sterile field.

The technologies disclosed in the Summate patents may be used to obtain information about the usage of non-sterile items. For example, as disclosed therein, a surgical item may be located within a well in a surgical supply tray. A machine-readable object (e.g., an RFID tag) may be located near the well. When the item is removed from the tray, a wand may be used to read information about the item from the machine-readable object located near the well from which the item was removed. Typically such a wand is coupled to a second computer in the non-sterile field. The information that is read from the machine-readable object may be stored in the second computer, such as in application software executing on the second computer. The Summate patents disclose a variety of other embodiments for recording item usage.

As the description above illustrates, in prior art systems, there are two separate workflows for capturing and storing data about sterile items and non-sterile items. Embodiments of the present invention, in contrast, use a single workflow to capture and store data about both sterile items and non-sterile items. Such a single workflow may, for example, use a single computer to capture and store data about both sterile items and non-sterile items. Even more specifically, such a single workflow may, for example, use a single software application to capture and store data about both sterile items and non-sterile items. Such a workflow may, for example, store the data about both the sterile items and the non-sterile items in a single data store. The capturing of item usage data may occur, for example, either within or at the edge of the sterile field.

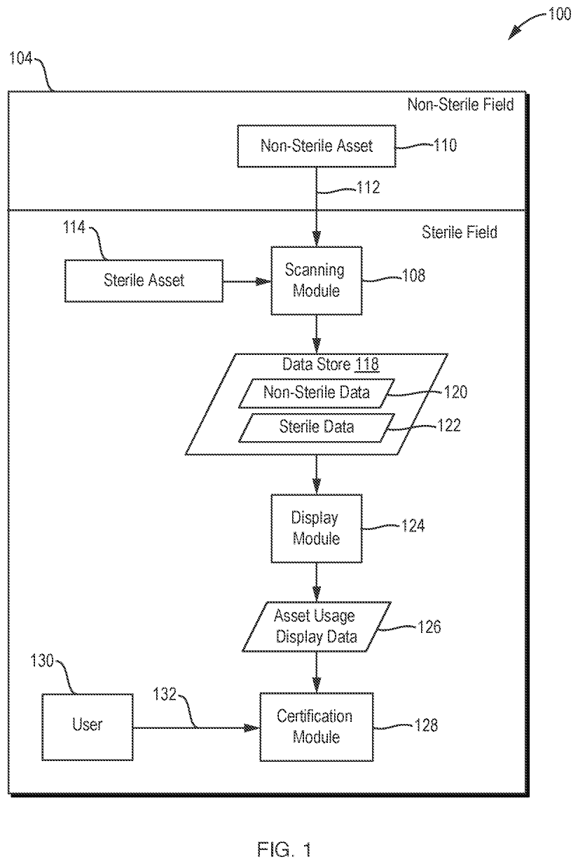

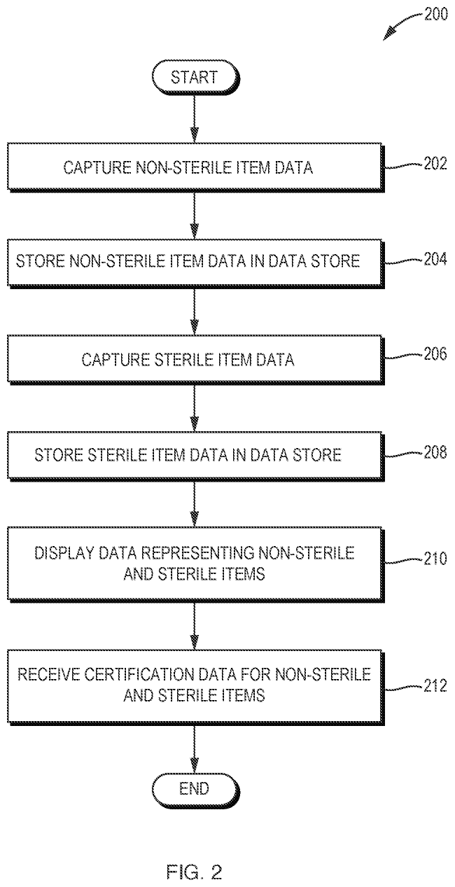

For example, referring to FIG. 1, a diagram is shown of a system 100 for capturing data representing usage of sterile and non-sterile objects in a surgical field according to embodiment of the present invention. Referring to FIG. 2, a flowchart is shown of a method 200 performed by the system 100 of FIG. 1 according to embodiment of the present invention.

The surgical field 100 may include both a sterile field 102 and a non-sterile field 104. FIG. 1 also illustrates a boundary 106 between the sterile field 102 and the non-sterile field 104, also referred to herein as the edge 106 of the sterile field 102.

The system 100 also includes a scanning module 108, which may, for example, include a computer and one or more scanning components, such as any one or more of the following, in any combination: a barcode reader, an RFID tag reader, and an image capture device (e.g., camera). The scanning module 108 may also include software, executing on the computer, for controlling the scanning components to perform the scanning functions disclosed herein and for performing other functions disclosed herein, such as for storing and displaying item usage data.

Although the scanning module 108 is shown in FIG. 1 as being at the edge 106 of the sterile field, this is merely an example and not a limitation of the present invention. The scanning module 108 may, for example, be in the non-sterile field 104 or the sterile field 102. As another example, the scanning module 108 may be at the point of use of the non-sterile item 110 and/or sterile item 114, where such point of use may, for example, be at the edge of the sterile field, within the sterile field, or within the non-sterile field. As another example, the scanning module 108 may change locations over time and be at different locations when performing different functions disclosed herein. Alternatively, for example, the scanning module 108 may stay in the same location while performing all functions disclosed herein.

The system 100 also includes one or more non-sterile items 110 and one or more sterile items 114. Although the non-sterile items 110 are shown in FIG. 1 as being within the non-sterile field 110 and the sterile items 114 are shown as being within the sterile field 102, this is merely an example and not a limitation of the present invention.

The scanning module 108 captures data 112 about one of the non-sterile items 110 (FIG. 2, operation 202). The scanning module 108 may capture the data 112 in any of a variety of ways. In general, the scanning module 108 may capture the data 112 by reading machine readable data from: (1) the non-sterile item 110; or (2) a machine-readable object associated with (e.g., coupled to or in proximity to) the non-sterile item 110. For example, the scanning module 108 may capture the data 112 by reading the data 112 from a barcode (e.g., using a barcode reader or image capture device) or RFID tag (e.g., using an RFID tag reader) associated with the non-sterile item 110.

The scanning module 108 may store the captured data 112 about the non-sterile item 110 in a data store 118 as non-sterile data 120 (FIG. 2, operation 204). The stored non-sterile data 120 may include, for example, the data 112 read from the non-sterile item 110 and/or data derived from the data 112 read from the non-sterile item 110 (such as data obtained from an additional data source based on the data 112). Although the data store 118 is shown outside the scanning module 108 in FIG. 1, the data store 118 may be within the scanning module. For example, the scanning module 108 may include a computer, and the data store 118 may be within the computer. For example, the computer may execute a software application, and the software application may store the non-sterile data 120 in the data store 118 within the computer.

The scanning module 108 captures data 116 about one of the sterile items 114 (FIG. 2, operation 206). The scanning module 108 may capture the data 116 in any of a variety of ways. In general, the scanning module 108 may capture the data 116 by reading machine readable data from: (1) the sterile item 114; and/or (2) a machine-readable object associated with (e.g., coupled to or in proximity to) the sterile item 114. For example, the scanning module 108 may capture the data 116 by reading the data 116 from a barcode (e.g., using a barcode reader or image capture device) or RFID tag (e.g., using an RFID tag reader) associated with the sterile item 114.

As part of, or after, the data capture operation 206, the system 100 (e.g., the scanning module 108) may determine whether the scanned item has been recalled or expired. Such a determination may be based on, for example: (1) the data captured in the data capture operation 206 by the scanning module 108; and/or (2) data obtained by the system 100 from one or more sources other than the scanning module 108 (such as one or more databases containing information about item recall dates and/or item expiration dates). Any such combination of (1) and (2) is referred to herein as “item date data.”

The system 100 may, for example, determine whether the item date data includes a recall date for the scanned item and, if the item date data is determined to include such a recall date, the system 100 may determine whether the recall date is earlier than the current date (i.e., the date on which the data capture operation 206 is performed). If the system 100 determines that the recall date is earlier than the current date, then the system 100 may, in response to and/or based on that determination, generate output to a user of the system 100 indicating that the scanned item has been recalled.

Similarly, as another example, the system 100 may determine whether the item date data includes an expiration date associated with the scanned item and, if the item date data is determined to include such an expiration date, the system 100 may determine whether the expiration date is earlier than the current date (i.e., the date on which the data capture operation 206 is performed). If the system 100 determines that the expiration date is earlier than the current date, then the system 100 may, in response to and/or based on that determination, generate output to a user of the system 100 indicating that the scanned item has expired.

The scanning module 108 may, for example, use a single hardware module (e.g., barcode reader or RFID tag reader) to capture both the non-sterile data 112 from the non-sterile item 110 and to capture the sterile data 116 from the sterile item 114. The scanning module 108 may, for example, use a single software module (e.g., the software application mentioned above) to capture both the non-sterile data 112 from the non-sterile item 110 and to capture the sterile data 116 from the sterile item 114.

The scanning module 108 may store the captured data 116 about the sterile item 114 in the data store 118 as sterile data 122 (FIG. 2, operation 208). The stored non-sterile data 120 may include, for example, the data 116 read from the sterile item 114 and/or data derived from the data 116 read from the sterile item 114 (such as data obtained from an additional data source based on the data 116). As illustrated in FIG. 1, the same data store 118 (e.g., in a single computer) may store both the non-sterile data 120 and the sterile data 122.

Although the description above refers to capturing and storing data about only a single non-sterile item and a single sterile item, the same operations may be repeated for any number of sterile and non-sterile items. In fact, the system 100 and method 200 may be used to capture data, using the techniques described above, about all items used within a particular surgery, so that the resulting data store 118 contains data about all items used during the particular surgery. The data 120 and 122 in the data store 118 are examples of what are referred to herein as “item usage data.”

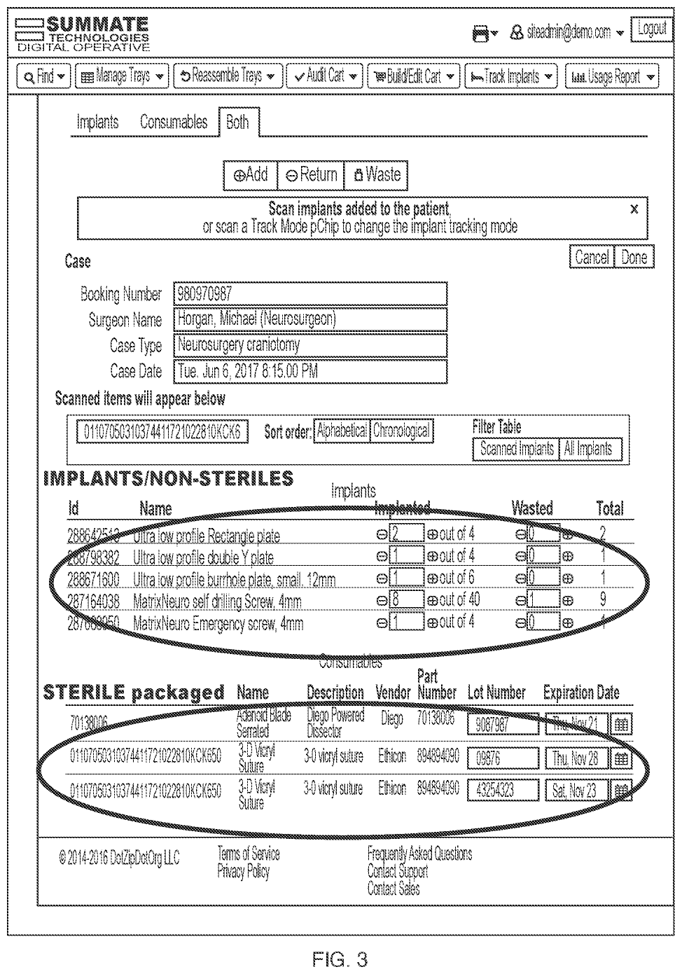

The system 100 also includes a display module 124, which may, for example, be within the same computer as the scanning module 108. For example, a single software application on that computer may include the scanning module 108, data store 118, and display module 124. The display module 124 may generate and output to a user 130 of the system 100, on an output device (e.g., display monitor), item usage display data 126 based on and representing some or all of the non-sterile data 120 and sterile data 122 (FIG. 2, operation 210). An example of such a user interface is shown in FIG. 3. The item usage display data 126 may, for example, include item usage data about one or more of the non-sterile items 110 and one or more of the sterile items 114. The display module 124 may display at least some of the non-sterile data 120 and at least some of the sterile data 122 simultaneously. As a result, a user 130 of the system 100 may view at least some of the non-sterile data 120 and at least some of the sterile data 122 simultaneously.

The user 130 may review the item usage display data 126 to verify that the data 126 accurately reflects the non-sterile items 110 and the sterile items 114 whose information was captured by the scanning module 108. The user 130 may provide certification input 132 to a certification module 128 in the system 100 (FIG. 2, operation 212). The certification module 128 may, for example, be a software module, such as a software module in the same software application executing on the same computer as the scanning module and display module 124. The certification input 132 may, for example, be binary input indicating whether or not the user 130 approves of (certifies) the item usage display data as accurately reflecting the non-sterile items 110 and the sterile items 114 whose information was captured by the scanning module 108. The certification input 132 may be a single input, such as a single keystroke or a single selection of a single user interface element (e.g., button or checkbox), that applies to all of the item usage display data 126, including portions of the item usage display data 126 that represent non-sterile items 110 and portions of the item usage display data 126 that represent sterile items 114.

Embodiments of the present invention may use the certification input 132 to perform any of a variety of functions. For example, the system 100 may send the certification data 132 to one or more external systems for storage and/or other processing, such as one or more of Electronic Health Record (EHR) software, Enterprise Resource Planning (ERP) software, Inventory Management System software, and any other kind of operation room information system (e.g., clinical documentation system, enterprise data system, coding/billing system). For example, such external systems may receive and store the certification data 132 in association with an EHR of the patient on whom the scanned item was used. In some embodiments, such external systems may store store some or all of the non-sterile data 120 and the sterile data 122 only if the certification input 132 indicates that the user 130 has certified the item usage display data 126 as being accurate.

Embodiments of the present invention have a variety of advantages. For example, embodiments of the present invention include computer-automated methods and systems for capturing data about the use of surgical items at the point of use, e.g., within or at the edge of the sterile field. The ability to capture item usage data at the point of use both decreases the amount of effort required to perform such capture and decreases the likelihood of error in the record of such item usage. For example, even in the case of item use capture which previously was performed automatically, moving the location of asset capture to the point of use (e.g., from outside the sterile field to the edge of, or inside, the sterile field) increases the likelihood that each use of an item will be captured and that such capture will be performed accurately. Furthermore, in the case of item use capture which previously was performed manually, automating such item use capture and performing such item use capture at the edge of, or inside, the sterile field both decreases the amount of manual effort required to perform such capture and increases the likelihood that such capture will be performed accurately.

Furthermore, embodiments of the present include computer-automated methods and systems which capture data about the use of all items that are used within a surgery. This differs from existing practice, in which either all item usage capture is performed manually, or in which computer-automated methods and systems are used to capture data about some, but not all, of the items that are used within a surgery. The ability of embodiments of the present invention to create a complete record of all items used in a surgery enables invoices and the patient's health record to reflect the full set of items that were used during the patient's surgery.

Furthermore, embodiments of the present invention implement a single workflow for computer-implemented capture of item usage data during a surgery, including capture of data about the usage of both sterile and non-sterile assets. This differs from existing techniques, in which one workflow is used to capture data about sterile items and another workflow is used to capture data about non-sterile assets. Such a unified workflow is both simpler for the human operator to carry out and requires fewer computing resources to implement. For example, such a single workflow may involve using a single computer (rather than two or more computers) to capture the item usage data. As another example, item usage data about both sterile and non-sterile items may be captured by a single software application executing on a single computer, rather than by multiple software applications executing on one or more computers. As yet another example, item usage data about both sterile and non-sterile items may be captured and stored into a single unified data store (e.g., within a single software application), rather than in two or more data stores. Such unification of the item usage data workflow thereby provides improvements in efficiency in multiple ways.

Furthermore, embodiments of the present invention may present information representing item usage data via a single user interface. For example, embodiments of the present invention may present information representing item usage data about both sterile and non-sterile items via a single user interface of a single software application, such as by simultaneously displaying both such kinds of data to the user 130 on a screen. The item usage data presented in the single user interface may include item usage data about all items that were used in the corresponding surgery. This differs significantly from existing techniques, in which item usage data about sterile and non-sterile items are not presented in a single user interface, but instead are, at best, displayed in separate user interfaces, such as by displaying item usage data about sterile items in a first user interface of a first software application and displaying item usage data about non-sterile items in a second user interface of a second software application, where the first and second software applications may execute on first and second computers, respectively. Presenting item usage data about both sterile and non-sterile items in a single user interface has a variety of benefits, including making it possible for the user 130 to approve of (e.g., certify) the item usage data for all items (e.g., both sterile items and non-sterile assets) by providing a single user input (e.g., by selecting a single “approve” button in the user interface).

One embodiment is directed to a method comprising: (1) scanning, at a scanning module, sterile item usage data associated with a sterile item during surgery at a point of use; (2) scanning, at the scanning module, non-sterile item usage data associated with a non-sterile item during surgery at the point of use; (3) storing the sterile item usage data and the non-sterile item usage data in a data store; and (4) displaying item usage display data, derived from the sterile item usage data and the non-sterile item usage data.

Scanning the sterile item usage data may include scanning the sterile item usage data from a machine-readable object associated with the sterile item to obtain the sterile item usage data. The machine-readable object associated with the sterile item may include a bar code, and scanning the machine-readable object associated with the sterile item may include scanning the bar code. The machine-readable object associated with the sterile item may include an RFID tag, and wherein scanning the machine-readable object associated with the sterile item comprises scanning the bar code. The machine-readable object associated with the sterile item may be coupled to the sterile item.

The machine-readable object associated with the sterile item may be in an item tray containing the sterile item. The machine-readable object associated with the sterile item may be coupled to packaging containing the sterile item.

Scanning the non-sterile item usage data may include scanning the non-sterile item usage data from a machine-readable object associated with the non-sterile item to obtain the non-sterile item usage data. The machine-readable object associated with the non-sterile item may include a bar code, and scanning the machine-readable object associated with the non-sterile item may include scanning the bar code. The machine-readable object associated with the non-sterile item may include an RFID tag, and scanning the machine-readable object associated with the non-sterile item may include scanning the bar code. The machine-readable object associated with the non-sterile item may be coupled to the sterile item. The machine-readable object associated with the non-sterile item may be coupled to packaging containing the sterile item. The machine-readable object associated with the non-sterile item may be in an item tray containing the sterile item.

Scanning the sterile item usage data may include using an image capture device to capture an image of the sterile item to obtain the item usage data associated with the sterile item. Scanning the non-sterile item usage data may include using an image capture device to capture an image of the non-sterile item to obtain the item usage data associated with the non-sterile item.

The data store may be contained within at least one non-transitory computer-readable medium. The data store may be contained within a single computer-readable storage device. The data store may be contained within a single computer.

The scanning module may be coupled to a computer, and the computer may cooperate with the scanning module to perform (1) and (2). Step (3) may be performed by the computer. Step (4) may be performed by the computer. The computer may execute a software application, and the software application may cooperate with the scanning module to perform (1) and (2). The software application may control the computer to perform (3). The software application may control the computer to perform (4). The software application may control the computer to perform (4), and (4) may include contemporaneously displaying, in a single user interface: (1) item usage display data derived from the sterile item usage data, and (2) item usage display data derived from the non-sterile item usage data.

The method may further include: (5) obtaining, based on the item usage data associated with the sterile item, additional data associated with the sterile item; and (6) storing the additional data associated with the sterile item in the data store.

The method may further include: (5) obtaining, based on the item usage data associated with the non-sterile item, additional data associated with the non-sterile item; and (6) storing the additional data associated with the non-sterile item in the data store.

The method may further include: (5) receiving, from a user, certification input indicating whether the user certifies at least some of the displayed item usage display data as accurate.

Another embodiment is directed to a system including: (1) a scanning module, which includes first scanning means for scanning sterile item usage data associated with a sterile item during surgery at a point of use; and a second scanning means for scanning non-sterile item usage data associated with a non-sterile item during surgery at the point of use; (2) at least one non-transitory computer-readable medium having a data store stored thereon; (3) a data storage module for storing the sterile item usage data and the non-sterile item usage data in the data store; (4) means for deriving item usage display data from the sterile item usage data and the non-sterile item usage data; and (5) a display module for displaying the item usage display data.

The system may further include a machine-readable object associated with the sterile item; the first scanning means may include means for scanning the sterile item usage data from the machine-readable object associated with the sterile item to obtain the sterile item usage data.

The system may further include: a machine-readable object associated with the non-sterile item; the second scanning means comprises means for scanning the non-sterile item usage data from the machine-readable object associated with the non-sterile item to obtain the non-sterile item usage data.

The first scanning means may include a first image capture device for capturing an image of the sterile item to obtain the item usage data associated with the sterile item. The second scanning means may include a second image capture device for capturing an image of the non-sterile item to obtain the item usage data associated with the non-sterile item.

The system may further include a computer-readable storage device, and wherein the computer-readable storage device comprises the data store. The system may further include a computer, and wherein the computer comprises the computer-readable storage device and the data store.

The system may further include: a computer, comprising a processor and a non-transitory memory, coupled to the first scanning means and the second scanning means; wherein the non-transitory memory includes computer program instructions which, when executed by the processor, cause the processor to: (1) control the first scanning means to scan the sterile item usage data associated with the sterile item; and (2) control the second scanning means to scan the non-sterile item usage data associated with the non-sterile item.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention include features which are only possible and/or feasible to implement with the use of one or more machines, such as computers, computer processors, and/or other elements of a computer system. Such features are either impossible or impractical to implement mentally and/or manually. For example, embodiments of the present invention read data from a machine-readable object, such as by using a wand to read data from a chip. This function cannot be performed by a human manually or mentally.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic discs such as internal hard discs and removable discs; magneto-optical discs; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disc (not shown) or a removable disc. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

1. A method comprising:
(1) scanning, at a scanning module, sterile item usage data associated with a sterile item during surgery at a point of use;
(2) scanning, at the scanning module, non-sterile item usage data associated with a non-sterile item during surgery at the point of use;
(3) storing the sterile item usage data and the non-sterile item usage data in a data store; and
(4) displaying item usage display data, derived from the sterile item usage data and the non-sterile item usage data.

2. The method of claim 1, wherein scanning the sterile item usage data comprises scanning the sterile item usage data from a machine-readable object associated with the sterile item to obtain the sterile item usage data.

3. The method of claim 2, wherein the machine-readable object associated with the sterile item comprises a bar code, and wherein scanning the machine-readable object associated with the sterile item comprises scanning the bar code.

4. The method of claim 2, wherein the machine-readable object associated with the sterile item comprises an RFID tag, and wherein scanning the machine-readable object associated with the sterile item comprises scanning the bar code.

5. The method of claim 2, wherein the machine-readable object associated with the sterile item is coupled to the sterile item.

6. The method of claim 1, wherein the machine-readable object associated with the sterile item is in an item tray containing the sterile item.

7. The method of claim 2, wherein the machine-readable object associated with the sterile item is coupled to packaging containing the sterile item.

8. The method of claim 1, wherein scanning the non-sterile item usage data comprises scanning the non-sterile item usage data from a machine-readable object associated with the non-sterile item to obtain the non-sterile item usage data.

9. The method of claim 8, wherein the machine-readable object associated with the non-sterile item comprises a bar code, and wherein scanning the machine-readable object associated with the non-sterile item comprises scanning the bar code.

10. The method of claim 8, wherein the machine-readable object associated with the non-sterile item comprises an RFID tag, and wherein scanning the machine-readable object associated with the non-sterile item comprises scanning the bar code.

11. The method of claim 8, wherein the machine-readable object associated with the non-sterile item is coupled to the sterile item.

12. The method of claim 8, wherein the machine-readable object associated with the non-sterile item is coupled to packaging containing the sterile item.

13. The method of claim 8, wherein the machine-readable object associated with the non-sterile item is in an item tray containing the sterile item.

14. The method of claim 1, wherein scanning the sterile item usage data comprises using an image capture device to capture an image of the sterile item to obtain the item usage data associated with the sterile item.

15. The method of claim 1, wherein scanning the non-sterile item usage data comprises using an image capture device to capture an image of the non-sterile item to obtain the item usage data associated with the non-sterile item.

16. The method of claim 1, wherein the data store is contained within at least one non-transitory computer-readable medium.

17. The method of claim 16, wherein the data store is contained within a single computer-readable storage device.

18. The method of claim 1, wherein the data store is contained within a single computer.

19. The method of claim 1, wherein the scanning module is coupled to a computer, and wherein the computer cooperates with the scanning module to perform (1) and (2).

20. The method of claim 1, wherein (3) is performed by the computer.

21. The method of claim 20, wherein (4) is performed by the computer.

22. The method of claim 19, wherein the computer executes a software application, and wherein the software application cooperates with the scanning module to perform (1) and (2).

23. The method of claim 22, wherein the software application controls the computer to perform (3).

24. The method of claim 23, wherein the software application controls the computer to perform (4).

25. The method of claim 22, wherein the software application controls the computer to perform (4), and wherein (4) comprises contemporaneously displaying, in a single user interface: (1) item usage display data derived from the sterile item usage data, and (2) item usage display data derived from the non-sterile item usage data.

26. The method of claim 1, further comprising:
(5) obtaining, based on the item usage data associated with the sterile item, additional data associated with the sterile item; and
(6) storing the additional data associated with the sterile item in the data store.

27. The method of claim 1, further comprising:
(5) obtaining, based on the item usage data associated with the non-sterile item, additional data associated with the non-sterile item; and
(6) storing the additional data associated with the non-sterile item in the data store.

28. The method of claim 1, further comprising:
(5) receiving, from a user, certification input indicating whether the user certifies at least some of the displayed item usage display data as accurate.

29. A system comprising:
a scanning module comprising:
first scanning means for scanning sterile item usage data associated with a sterile item during surgery at a point of use; and
second scanning means for scanning non-sterile item usage data associated with a non-sterile item during surgery at the point of use;
at least one non-transitory computer-readable medium having a data store stored thereon;
a data storage module for storing the sterile item usage data and the non-sterile item usage data in the data store;
means for deriving item usage display data from the sterile item usage data and the non-sterile item usage data; and
a display module for displaying the item usage display data.

30. The system of claim 29, further comprising:
a machine-readable object associated with the sterile item; and
wherein the first scanning means comprises means for scanning the sterile item usage data from the machine-readable object associated with the sterile item to obtain the sterile item usage data.

31. The system of claim 29, further comprising:
a machine-readable object associated with the non-sterile item; and
wherein the second scanning means comprises means for scanning the non-sterile item usage data from the machine-readable object associated with the non-sterile item to obtain the non-sterile item usage data.

32. The system of claim 29, wherein the first scanning means comprises a first image capture device for capturing an image of the sterile item to obtain the item usage data associated with the sterile item.

33. The system of claim 29, wherein the second scanning means comprises a second image capture device for capturing an image of the non-sterile item to obtain the item usage data associated with the non-sterile item.

34. The system of claim 29, further comprising a computer-readable storage device, and wherein the computer-readable storage device comprises the data store.

35. The system of claim 34, further comprising a computer, and wherein the computer comprises the computer-readable storage device and the data store.

36. The system of claim 29, further comprising:
a computer, comprising a processor and a non-transitory memory, coupled to the first scanning means and the second scanning means; and
wherein the non-transitory memory includes computer program instructions which, when executed by the processor, cause the processor to:
control the first scanning means to scan the sterile item usage data associated with the sterile item; and
control the second scanning means to scan the non-sterile item usage data associated with the non-sterile item.
